(19) United States
(12) Patent Application Publication
(10) Pub. No.: US 2026/0078154 A1
(43) Pub. Date: Mar. 19, 2026

(54) System and Method for Redirecting Audio and Video Data Streams in a Display-Server Computing System

(71) Applicant: Netzyn, Inc., Richardson, TX (US)

(72) Inventor: Steve Bakke, Richardson, TX (US)

(73) Assignee: Netzyn, Inc., Richardson, TX (US)

(21) Appl. No.: 19/259,374

(22) Filed: Jul. 3, 2025

Related U.S. Application Data
(63) Continuation of application No. 16/982,966, now Pat. No. 11,897,924.
(60) Provisional application No. 62/646,429, filed on Mar. 22, 2018.

(51) Int. Cl. C07K 14/435; A61K 38/00; A61P 7/04; C07K 14/765

(52) U.S. Cl. CPC C07K 14/43527; A61P 7/04; C07K 14/765; A61K 38/00; C07K 2319/00

(57) ABSTRACT

A system operative on a data communications network to operate on an audio/video data includes a server computer communicatively connected to the network. The server computer includes an application unit running an application program. The application program is accessible to a user device communicatively connected to the network. The application unit requires the audio/video data to run the application program. The system also includes an audio/video server computer communicatively connected to the network. The audio/video server computer communicates the audio/video data over the network responsive to a request. The user device includes at least a buffer, a decoder and an output device. The need request is sent by the server computer over the network to the user device, for receipt of the audio/video data for operation of the application unit. The need request includes an identifier of the audio/video data and a network address of the audio/video server computer. The user device, upon receipt of the need request, requests communicatively over the network to the audio/video server computer and the audio/video server computer delivers communicatively over the network the audio/video data to the user device. The user device reformats the audio/video data and delivers the reformatted audio/video data to the server computer. The server computer reverses the reformatted audio/video data to obtain substantially the original audio/video data for operations of the application unit.

### CROSS-REFERENCE TO RELATED APPLICATION

The present application has benefit of priority of the following application, which is co-pending and has at least one same inventor of the present application and is incorporated herein by this reference: U.S. Provisional Patent Application Ser. No. 62/646,429, titled “System and Method for Redirecting Audio and Video Data Streams in a Display-Server Computing System,” filed Mar. 22, 2018.

### TECHNICAL FIELD

The present invention generally relates to computing systems and more specifically relates to display-server systems and communications networks.

### BACKGROUND

End-user computing software programs and their operations (i.e., applications) have traditionally been processed (i.e., run) on an end-user device itself, for example, a personal computer (PC) running an application of the PC, a smartphone running an application on the smartphone, a game console running a game application on the console, and others. A different approach is to run end-user applications on data center-located servers. These server-run applications are delivered to the end-user device, such as those described or even a display device with limited processing capability, via communicative connection of the end-user device to the servers. Such a display-server system is described in U.S. Pat. No. 8,700,723. When the application that is run on the server receives audio and video from an external source, the audio and video data must be passed on to the end-user display device by the server. The network bandwidth required includes bandwidth occupied by the communication of the data to the server and also bandwidth occupied by the communication of the data by the server to the end-user device. In effect, the bandwidth requirement is doubled.

Referring to FIG. 1, a non-exclusive example of a conventional system 100 includes an application unit 105 running directly on a device 106, such as an end-user computer, smart phone, console or other device. The device 106 is communicatively connected to a network 102. System 100 details typical usage of a non-display-server system in which significant processing and storage for operating the application unit is required of the device 106. The application unit 105 running on the device 106 requires audio/video data available from an audio/video server 101 communicatively connected to the network 102. The device 106 sends a request 103 for that audio/video information on the network 102 to the audio/video server 101. The audio/video server 101 responds with the desired audio/video data 113 communicated over the network 102 to the device 106. The application unit 105 of the device 106 receives the audio/video data 113 and stores it in a buffer 112 of the device 106. When there is sufficient audio/video data in the buffer 112, the application unit 105 issues a command 107 to a decoder 110 of the device 106 to start decoding. The output of the decoder is displayed on display 109.

In a display-server computing system, a server computer operates the application unit. When audio and/or video data is needed for operating the application unit, the server computer conventionally contacts an audio/video server computer via a network and requests the audio/video data. The audio/video data is sent over the network by the audio/video server computer to the server computer operating the application unit. The server computer operating the application unit then must send the audio/video data for operations of the application unit to a user device, such as a display, personal computer, smart phone, console or other device. The user device communicates with the server computer, and the server computer delivers the audio/video data associated with the operating application unit on the server computer. Bandwidth requirements are significant in such conventional display-server computing system, because audio/video data must be retrieved by the server computer and communicated by the server computer to the user device.

It would be a significant improvement in the art and technology to provide for more efficient and optimized flow of audio and video data to the end-user device, such as a personal computer, a smartphone, a console, or even a display with limited processing capability or other device.

### SUMMARY

An embodiment of the invention is a system including a server computer running an application unit on the server computer, a user device communicatively connected to that server computer, and another server computer capable of communicating audio and video data. The server computer of the application unit and the user device each include a decoder for decrypting the audio and video data.

The server computer running the application unit has need for audio/video data from the other server computer of the audio/video data for operation of the application unit (typically the other server computer of audio/video data differs from that running the application unit, although the same server may host the application unit and provide the audio/video data in embodiments). A request for that audio/video data together with an identifier of the audio/video server computer is sent to the user device by the server computer running the application unit. The user device forwards that request to the audio/video server computer. The audio/video server computer responds with the desired audio/video data to the user device. The user device receives the audio/video data and stores it in a buffer. The audio/video data is also reformatted by the user device to only include data that the server computer running the application unit needs for operations of the application unit, and does not include data that the decoder needs. This greatly reduces the audio/video data size of the reformatted data. The reformatted data is communicated by the user device to the server computer of the application unit for operating the application unit.

The server computer of the application unit receives the reformatted audio/video data and transforms the data back to a standard format before handing off the audio/video data to the application unit. To the application unit operated on the server computer, the audio/video data is indistinguishable from the original audio/video data sent from the audio/video server computer to the user device. The application unit operating on the server computer stores this audio/video data in a buffer and uses as normal for operating the application unit. When there is sufficient audio/video data in the buffer, the application unit issues a command to a decoder of the server computer to start decoding. The decode command is forwarded to a user device, such as a display device, and a decoder of the user device will start decoding the audio/video from the user device's local buffer. Therefore, the audio/video data used by user device's decoder is the actual audio/video data sent from the audio/video server and a very limited amount of audio/video data was sent to the server by the user device.

### BRIEF DESCRIPTION OF THE DRAWINGS

The present invention is illustrated by way of example and not limitation in the accompanying figures, in which like references indicate similar elements, and in which:

FIG. 1 illustrates a system operative over a data communications network of a server computer running an application program accessed by a user or display system in a conventional display-server system, according to certain embodiments of the invention;

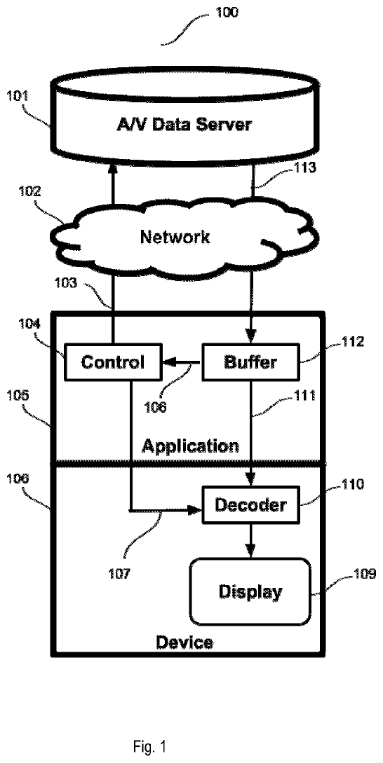

FIG. 2 illustrates a system operative over a data communication network of a server computer running an application program accessed by a user or display system, in which the user or display system, rather than the server computer, obtains audio/video data from an audio/video server computer, and the user or display system transmits reformatted audio/video data to the server computer for use by the running application program, according to certain embodiments of the invention; and

FIG. 3 illustrates exemplary audio/video data streams, including reformatted audio/video data streams and reconstructed audio/video data streams, delivered to and used by a user or display device and a server computer running an application program accessed by the user or display device, according to certain embodiments of the invention.

### DETAILED DESCRIPTION

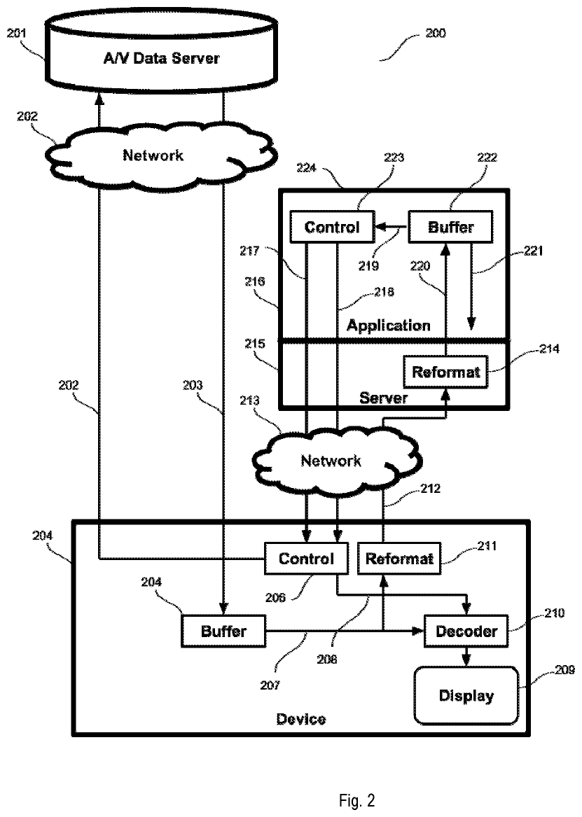

Referring to FIG. 2, a non-exclusive example embodiment of a system 200 includes a server computer 215. The server computer 215 includes or communicatively connects, such as over a data communications network, to an application unit 224. The server computer 215 includes, by way of example and not limitation, a processor 223, a buffer 222, and a reformatting module 214. The application unit 224 also includes a decoder. The application unit 224 runs on the server computer 215.

The server computer 215 of the application unit 224 communicatively connects to one or more network 202, 213, which may be the same or different network and may in each instance include combinations of communications networks, e.g., wired and/or wireless data networks.

An audio/video data server computer 201 is communicatively connected to one or more network 202, 213. In the example embodiment, the network 202 may be same or different from the network 213. As non-exclusive example, the audio/video server computer 201 may include a processor, a buffer, and memory. The audio/video data server computer 201 communicates over the network 202 data representing audio and video information that is useable by the application unit 224 of the server computer 215, and that is also useable by a user device 204, such as, for example, a display, a personal computer, a smartphone, a console, or other device, for output display or audio.

The user device 204 is communicatively connected via a network 202 to the audio/video server computer 201 and via a network 213 to the server computer 215. The networks 202, 213 may be same or different, in whole or part. The user device 204, as non-exclusive example, includes a control/processor 206, a buffer 204, a decoder 210, a display 209 or other output, and a reformat module 211. The user device 204 may include other or alternate elements, as applicable. The user device 204 may but need not necessarily have limited storage and processing capability. In the example embodiment, the application unit 224 operating on the server computer 215 is accessed by the user device 204 over the network 213 for providing a running application for the user device 204.

In operation, the server computer 215 controllingly operates the application unit 224 for access by the user device 204. In such operation, the application unit 224 requires audio/video data. The audio/video data is available from the audio/video data server computer 201. The server computer 215 communicatively sends on the network 213 a request 217 for that audio/video data, together with an identifier of the audio/video server 201, to the user device 204. The user device 204, via the processor 206, receives the request 217. The user device 204 communicatively forwards on the network 202 a request 202 to the audio/video server 201. The audio/video server 201 responds over the network 202 to the user device 204 with the desired audio/video data 203. The user device 204 receives the audio/video data 201 and stores it in a buffer 204. The audio/video data 203 is also reformatted 211 by the user device 204 to only include data that the application unit 224 needs for operation of the application unit 224, and does not include data that the decoder of the server computer 215 needs. The reformatted 211 audio/video data is sized significantly smaller than the original audio/video data received by the user device 204 from the audio/video server 201. The reformatted data 212 is sent by the user device 204 to the server computer 215 over the network 213.

The server computer 215 receives over the network 213 the reformatted data 212 from the user device 204. The server computer 215 processes the reformatted data 212 by transforming the data 212 back to standard format of the original audio/video data 203 received by the user device 204. The reformatting module 214 of the server computer 215 receives the reformatted data 212. The reformatting module 214 transforms the reformatted data 214 back to a standard format of an audio/video data 220, which is substantially the same as the original audio/video data 203. The transformed data, i.e., the audio/video data 220, from the reformatting module 214 is handed off by the reformatting module 214 to the application unit 224 of the server computer 215. The audio/video data 220 is indistinguishable to the application unit 224 from the original audio/video data 203 sent from the audio/video server 201. The application unit 224 stores the audio/video data 220 in the buffer 222 and the audio/video data 220 is used for operations by the application unit 224. When there is sufficient audio/video data 220 in the buffer 222, the application unit 224 issues a command 218 to the decoder of the server computer to start decoding the audio/video data 220. The decode command 218 is forwarded by the server computer 215 on the network 213 to the user device 204, and the decoder 210 of the user device 204 decodes the original audio/video data 203 stored in the user device's local buffer 204. The output of the decoder 210 is displayed on display 209, output as audio, or otherwise output by the user device 204 according to the nature of the audio/video data 203.

Therefore, the audio/video data 203 used by the decoder 210 is the actual audio/video data 203 sent by the audio/video server 201 to the user device 204. The audio/video data 212 sent by the user device 204 to the server computer 215 is significantly less than the original audio/video data 203. Bandwidth requirements for the communications are reduced.

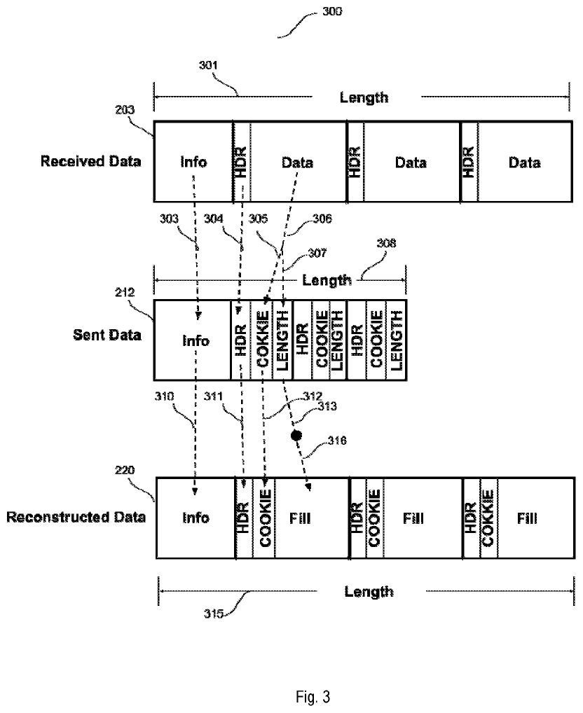

Referring to FIG. 3, in conjunction with FIG. 2, a non-exclusive example embodiment of a system 300 includes three data formats for audio/video data in the foregoing. The three data formats include a received audio/video data stream 203, a sent audio/video stream 220, and a reconstructed audio/video data stream 220. The received audio/video data stream 203 is received by the user device 204 from the audio/video server 201.

The user device 204 reformats the received audio/video data stream 203 transforming to the sent audio/video data stream 203. The reformating consists of copying info data 303 and header data 304 directly to sent audio/video data 212. Then the decoder data 306 is converted into a cookie 305 and a length 307. This process is repeated for each section of the received audio/video data stream 203.

The sent audio/video data 212 is received by the server computer 215 from the user device 204. The server computer 215 reformats 214 the sent audio/video data 212 from the user device 204 into the reconstructed audio/video data stream 220. The reformating consists of copying the info data 310, header data 311 and cookie 312 data directly to the reconstructed audio/video data stream 220. Then using the length 313, that amount of fill data 316 is created in the reconstructed audio/video data stream 220. This process is repeated for each section of the sent audio/video data 212.

The length 301 of the received audio/video data stream 203 and the length 301 of the reconstructed audio/video data stream 220 is exactly the same. The length 308 of the sent audio/video data stream 212 is much smaller than the received audio/video data stream 203.

In the foregoing specification, the invention has been described with reference to specific embodiments. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention.

Benefits, other advantages, and solutions to problems have been described above with regard to specific embodiments. However, the benefits, advantages, solutions to problems and device(s), connection(s) and element(s) that may cause any benefit, advantage, or solution to occur or become more pronounced are not to be construed as a critical, required, or essential feature or element of any or all the claims. As used herein, the terms “comprises, “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A system operating on a data communications network, comprising:
a server computer communicatively connected to the network;
an application unit of the server computer, the application unit of the server computer requires an audio/video data for operations;
a display device communicatively connected to the server computer over the network;
a reformatter unit of the display device; and
an audio/video server computer communicatively connected to the display device over the network, the audio/video server has a network identifier unique to the audio/video server;
wherein the application unit sends a request over the network to the display device for audio/video data, together with the network identifier of the audio/video server computer;
wherein the display device sends a request over the network to the audio/video server computer at the network address, for the audio/video data;
wherein the audio/video server computer sends the audio/video data associated with the request by the display device, to the display device over the network;
wherein the reformatter unit of the display device transforms the audio/video data to a reformatted audio/video data of small size than the audio/video data;
wherein the display device sends the reformatted audio/video data over the network to the application unit of the server computer.

2. The system of claim 1, wherein the reformatted audio/video data delivered to the application unit by the display device contains many time less bytes then the audio/video data.

3. The system of claim 1, wherein the server computer transforms the reformatted audio/video data to the audio/video data;
wherein the application unit operates with the audio/video data as transformed from the reformatted audio/video data.

4. The system of claim 1, wherein the display device decodes the audio/video data and outputs the audio/video data via either a display of the display device or another output device.

5. A system operative on a data communications network to operate on an audio/video data, comprising:
a server computer communicatively connected to the network, the server computer includes an application unit running an application program communicatively connected to the network, the application unit requires the audio/video data to run the application program;
an audio/video server computer communicatively connected to the network, the audio/video server computer communicates the audio/video data over the network responsive to a request; and
a user device communicatively connected to the network, the user device includes at least a buffer, a decoder and an output device;
wherein a need request is made by the server computer over the network to the user device, for receipt of the audio/video data for operation of the application unit, the need request includes an identifier of the audio/video data and a network address of the audio/video server computer;
wherein the user device, upon receipt of the need request, requests communicatively over the network to the audio/video server computer and the audio/video server computer delivers communicatively over the network the audio/video data to the user device.

6. The system of claim 5, further comprising:
a reformatter device of the user device, the reformatter device transforms the audio/video data to a reformatted audio/video data;
wherein the user device delivers the reformatted audio/video data over the network to the server computer.

7. The system of claim 6, further comprising:
a reverse reformat unit of the server computer, the reverse reformat unit transforms the reformatted audio/video data to substantially the audio/video data for operation of the application unit;
wherein the application unit operates on the substantially the audio/video data as if operating on the audio/video data.

8. The system of claim 7, further comprising:
a decoder of the user device, the decoder of the user device transforms the audio/video data to output by the user device; and
a decoder of the server computer, the decoder of the server computer transforms the substantially the audio/video data to run on the application unit.

9. The system of claim 6, wherein the byte size of the reformatted audio/video data is substantially less than the byte size of the audio/video data.

10. The system of claim 6, wherein reduced bandwidth is required for display or other output of the audio/video data by the user device and delivery of the audio/video data to the server computer for operation of the application unit.

11. A method, comprising:
delivering over a data communications network by a server computer a need request for an audio/video data, to a user device communicatively connected to the server computer by the network, the need request includes an identifier of an audio/video server computer communicatively connected to the user device by the network;
accessing by the user device over the network the audio/video server computer to obtain the audio/video data;
reformatting the audio/video data by the user device, to a reformatted audio/video data;
sending over the network by the user device the reformatted audio/video data, for receipt by the server computer;
reverse reformatting the reformatted audio/video data received by the server computer, to substantially the audio/video data; and
operating by the server computer on the substantially the audio/video data.

12. The method of claim 11, further comprising:
running an application unit by the server computer; and
wherein delivering is responsive to the application unit;
wherein operating includes the application unit processes the substantially the audio/video data with same result as if the audio/video data.

13. The method of claim 12, further comprising:
storing by the user device the audio/video data in a buffer;
decoding by the user device the audio/video data of the buffer; and
outputting by the user device via an output device an audio and/or video represented by the audio/video data.

14. The method of claim 13, further comprising:
storing by the server computer the substantially the audio/video data from reverse reformatting in a buffer;
decoding by the server device the substantially the audio/video data of the buffer; and
running the application unit by the server computer with input of the substantially the audio/video data, responsive to the decoding.

15. The method of claim 11, wherein the reformatted audio/video data is smaller in size than the audio/video data.

16. The method of claim 12, wherein the reformatted audio/video data is smaller in size than the audio/video data.

17. The method of claim 13, wherein the reformatted audio/video data is smaller in size than the audio/video data.

18. The method of claim 14, wherein the reformatted audio/video data is smaller in size than the audio/video data.
